(19) United States
(12) Patent Application Publication
(10) Pub. No.: US 2019/0252040 A1
(43) Pub. Date: Aug. 15, 2019

(54) DETECTION OF CANCER-SPECIFIC DIAGNOSTIC MARKERS IN GENOME

(71) Applicant: KOREA ADVANCED INSTITUTE OF SCIENCE AND TECHNOLOGY, Daegeon (KR)

(72) Inventors: Dong Ho CHO, Daejeon (KR); Gyu Bum HAN, Daejeon (KR); Hye In SEO, Daejeon (KR); Byung Chang CHUNG, Daejeon (KR)

(21) Appl. No.: 16/323,948

(22) PCT Filed: Feb. 14, 2017
(86) PCT No.: PCT/KR2017/001581

§ 371 Date: Feb. 7, 2019

(30) Foreign Application Priority Data
Nov. 8, 2016 (KR) 10-2016-0147935
Feb. 13, 2017 (KR) 10-2017-0019559

(51) Int. Cl. G16B 25/10; G16B 20/20; C12Q 1/6827; C40B 40/06; G16B 25/20; G16B 40/20; G16H 50/50

(52) U.S. Cl. CPC G16B 25/10; G16B 20/20; C12Q 1/6827; G16H 50/50; G16B 25/20; G16B 40/20; C40B 40/06

(57) ABSTRACT

The present invention relates to a method for detecting cancer-specific diagnostic markers in a genome and, more specifically, to a method for identifying the relationship between cancer and genomic variations and detecting cancer-specific genomic changes, thereby enabling highly accurate cancer-specific biomarkers to be detected.

### TECHNICAL FIELD

The present invention provides a technology deriving a cancer-specific diagnostic marker through sequencing analysis information of a cancer genome.

### BACKGROUND ART

The genome has been shown to exhibit specific changes depending on diseases. However, until now, genomic analysis studies have focused on a gene that accounts for only about 1.2% of the whole genome to synthesize proteins.

Gene-focusing studies have derived many results from bio-information analysis. However, it is clearly shown that results obtained from these gene-focusing studies have limitations in explaining a number of diseases, and thus it is required to conduct comprehensive and structural analysis on genomic portions other than genes, which are capable of complementing these limitations.

A genetic linkage analysis method for screening a disease diagnostic marker, which is being performed by many researchers, is mostly performed based on exome sequencing that analyzes gene expression (about 1% of the genome) or single nucleotide polymorphism in the genomic population (about 0.06% of the genome). Upon considering the current technology trends for disease diagnosis, studies are underway to find genes associated with a particular disease by using human-shared polymorphisms of a particular gene (single nucleotide polymorphism, copy number polymorphism) or using expression information of the whole gene group, and to study the function of genes.

In particular, development of diagnostic technologies using genetic characteristics, gene expression, and nucleotide polymorphism of individuals are actively being conducted.

However, most of the diagnostic technologies that have been conducted so far have a very limited number of target genes, thus causing a limitation that is applicable only to some specific diseases, and even in deriving the disease-diagnostic marker, most of the diagnostic technologies are conducted based on all genes and the resulting proteins, causing inaccuracy of the technologies.

International Publication No. 2014-052909 discloses a method for diagnosing a disease by simultaneously considering phenotypic information and genetic variation of individuals using database including diseases, clinical information, and genetic information. International Publication No. 2014-052909 provides a system for diagnosis of diseases by linking the nucleotide sequence variation of the gene range with the clinical information of the patient, which is shown to have relationship between disease and genetic information at a high resolution.

However, International Publication No. 2014-052909 determines diseases by using genomic variation and clinical information, but is limited to some genetic information, thus causing limitations in analysis of whole genomic information. In addition, the above document uses a simple structure that confirms importance by assigning a truth value to each nucleotide sequence variation in disease diagnosis classification algorithm, and thus it is difficult to perform precise diagnosis through combination of multiple nucleotide sequence variations.

### RELATED ART DOCUMENT

(Patent Document 1) International Publication No. WO 2014-052909 (Published on Jul. 30, 2015)

### DISCLOSURE

### Technical Problem

An object of the present invention is to provide a method for detecting a cancer-specific diagnostic marker capable of analyzing cancer-specific genomic changes to identify a relationship between cancer and genomic variations and having high accuracy.

### Technical Solution

In one general aspect, a method for detecting a cancer-specific diagnostic marker performed in a program form executed by an arithmetic processing unit including a computer, includes: inputting whole genome sequence of a cancer sample and a normal sample; comparing and/or contrasting the whole genome sequencing information and the reference genome to obtain analyzed information; deriving a disease classification ratio from the analyzed information and sample information; constructing a library with respect to cancer-specific nucleotide sequence from the whole genome sequencing information of the cancer sample and the normal sample using the disease classification ratio; and deriving classification accuracy according to the disease classification ratio and a change of the number of variations from the constructed library.

### Advantageous Effects

The method for detecting a cancer diagnostic marker according to the present invention may perform analysis of genomic variants and variant positions shown in cancer genomes and normal genomes compared to reference genome information by using genome sequence obtained from actual cancer patients and normal patients, thereby detecting a cancer-specific diagnostic marker through determination of cancer-specific complex genomic information.

In addition, the cancer-specific diagnostic marker is capable of being detected by analyzing known cancer genomes other than genome sequence obtained from actual cancer patients and normal patients to determine cancer-specific complex genomic information.

In addition, it is possible to easily analyze complex variations by using a library constructed based on variation information and position information of the genome sequence, thereby detecting the cancer-specific diagnostic marker with high accuracy.

Further, the cancer diagnostic marker detected according to the present invention is easily applicable to all fields of medical fields and pharmaceutical fields such as bio-chips, precision diagnostic systems, kits, medical devices, and the like.

### DESCRIPTION OF DRAWINGS

FIG. 1 is a diagram illustrating reference genomic information and whole genome sequence of a sample used in a method for detecting a cancer diagnostic marker according to the present invention.

FIG. 2 is a diagram illustrating result information analyzed by comparing and/or contrasting reference genomic information and whole genome sequence of a sample in the method for detecting a cancer diagnostic marker according to the present invention.

FIG. 3 is a diagram illustrating a genome segmentation process in a target range extraction process in the method for detecting a cancer diagnostic marker according to the present invention.

FIG. 4 is a diagram illustrating library construction in the method for detecting a cancer diagnostic marker according to the present invention.

FIG. 5 is a flowchart illustrating an embodiment of the method for detecting a cancer diagnostic marker according to the present invention.

FIG. 6 is a diagram illustrating diagnosis of cancer for an arbitrary sample using a marker detected by the method for detecting a cancer diagnostic marker according to the present invention.

### BEST MODE

Hereinafter, the present invention is described in detail.

The terms used herein should be interpreted as generally understood by those skilled in the art unless otherwise defined.

The drawings and embodiments of the present specification are provided for those skilled in the art to easily understand and practice the present invention, and thus the present invention is not limited to the drawings and embodiments. In the drawings and the embodiments, contents that may obscure the gist of the invention may be omitted or exaggerated.

The present invention relates to a method for deriving or detecting a cancer-specific diagnostic marker based on genomic information analysis.

According to the present invention, it is possible to compare, analyze, and determine general life phenomenon and disease-related genome information based on the whole genome sequencing data, facilitate understanding of genome function, and detect an accurate cancer diagnostic marker.

In order to derive the cancer-specific diagnostic marker in the present invention, an information communication technology such as a big data processing technique, or the like, is applied to a vast amount of genome information to perform storage, translation, analysis and determination of genome information.

The method for detecting a cancer diagnostic marker according to the present invention generally proceeds as follows. First, information on the full-length genome (whole genome) sequence for cancer and normal sample (specimen) is obtained, and analysis information including genomic variation and position information of a cancer sample and a normal sample based on reference genome is obtained. Through the obtained analysis information, a library including the genomic variation and position information expected to be a cancer-specific genome change is constructed. A cancer-specific diagnostic marker is derived from the constructed library analysis.

More specifically, the method for detecting a cancer diagnostic marker according to the present invention is described as follows.

The present invention provides a method for detecting a cancer-specific diagnostic marker performed in a program form executed by an arithmetic processing unit including a computer, the method including: inputting whole genome sequence of a cancer sample and a normal sample; comparing and/or contrasting the whole genome sequence and the reference genome to obtain analyzed information; deriving a disease classification ratio from the analyzed information and sample information; constructing a library with respect to cancer-specific nucleotide sequence from the whole genome sequence of the cancer sample and the normal sample using the disease classification ratio; and deriving classification accuracy according to the disease classification ratio and a change of the number of variations from the constructed library.

Hereinafter, each step is described in detail below.

Inputting the whole genome sequence of the cancer sample and normal sample is described in detail.

In the inputting of the whole genome sequence of the cancer sample and normal sample, it is possible to secure information regarding the whole genome of the cancer sample and the normal sample.

The whole genome sequence of the cancer sample and the normal sample may be obtained from the genetic information database and may be obtained through the whole genome sequence information provided for each disease from authentication by the Cancer Genome Atlas (TCGA) of the National Institutes of Health (NIH). In addition, a sample of the actual patient taken in the hospital or directly may be requested from the sequencing company to obtain the full genome sequencing information of the sample. Alternatively, in some cases, the whole exome sequence may be obtained for an exome set that directly acts to synthesize proteins in the gene.

The whole genome sequence of the samples may be partially changed depending on the genetic information database, sequencing apparatus, sequencing method, and the like.

It is preferable that the obtaining of the whole genome sequence is based on human genome map information revealed from the human genome project.

The whole genome sequence of the cancer sample and the normal sample is information based on the method for detecting a cancer diagnostic marker according to the present invention, and subsequent processes proceed based on the difference in the genome properties of the samples included in the whole genetic sequencing information.

Among the information included in the whole genome information, particularly, chromosome information, in-chromosome variant positions, genomic variants, and reliability information may be used as important information in detection of cancer diagnostic marker.

Analysis of the information included in the whole genome sequence may be performed with addition or subtraction of information according to a program used for information analysis.

Comparing and/or contrasting the whole genome sequence and the reference genome sequence to obtain analysis information is described in detail.

The comparing and/or contrasting of the whole genome sequence and the reference genome sequence to obtain analysis information may obtain specific information included in the genomes of the samples. For example, there are information on variation of a genomic nucleotide sequence commonly found in a cancer sample and a combination thereof, information on a variation of a genomic nucleotide sequence commonly found in a normal sample and a combination thereof, information on variations in the genome sequence commonly shown and combinations thereof, information on genome sequences commonly shown to all of cancer samples, normal samples, and reference genomes, and the like.

The reference genome sequence may be obtained from the human genome map information obtained from the human genome project, and basically includes the chromosome, the position of the nucleotide sequence in the chromosome, and the nucleotide sequence.

Through analysis of the whole genome sequence and the reference genome, it is possible to obtain quality with respect to the chromosome information with nucleotide sequence variation in the genome of the cancer sample and the normal sample, the position information of the nucleotide sequence in the chromosome, the nucleotide sequence of the reference genome, the nucleotide sequence of the sample genome, and each nucleotide sequence, and these information may be used as important information for detecting a cancer diagnostic marker.

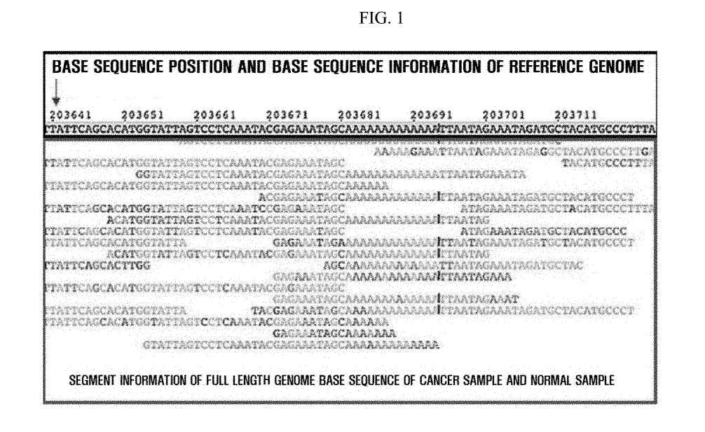

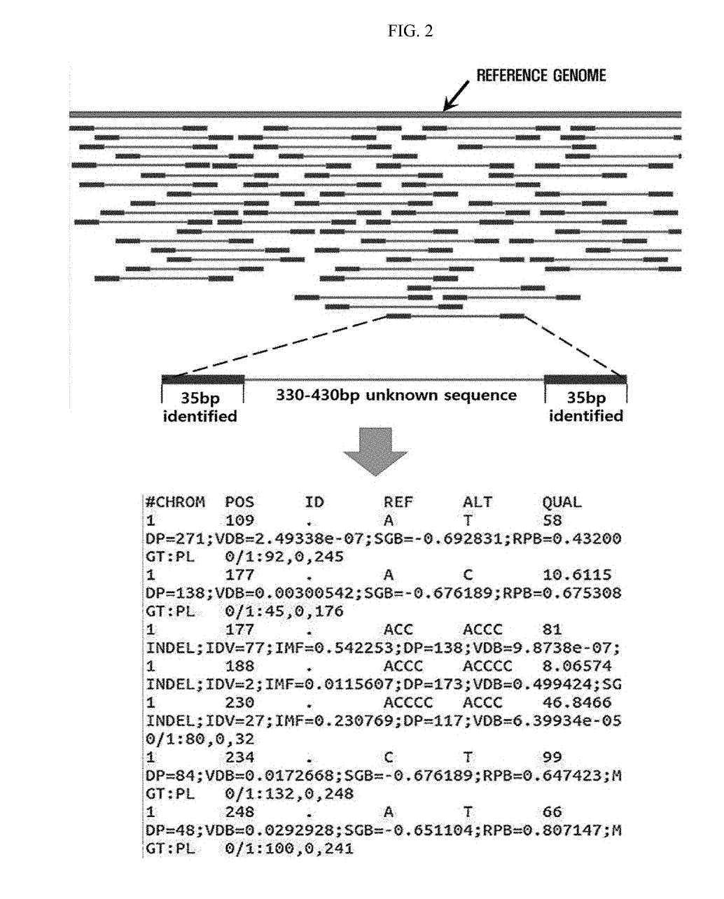

The whole genome sequence is in a form (see FIGS. 1 and 2) in which nucleotide sequence fragments are aligned on the basis of a reference genome, and thus it is not possible to analyze the genome only with this form itself. Thus, the analysis of the whole genome sequence and the reference genomic sequence information may be performed using a genome analysis program. For example, open source programs such as sequence alignment/map (SAM) tools and BCF tools, and the like, may be used. Depending on the type of program, processing and analysis results of the data may be different, and thus in the present specification, the nucleotide sequence and the base may be substituted with each other.

The analyzed information may be converted, stored, and managed into a predetermined platform, i.e., in the same frame form.

Among the analyzed information, the chromosome information (#CHROM), in-chromosome nucleotide sequence (nucleotide) position information (POS), reference genome sequence (nucleotide) information (REF), sample genome sequence (nucleotide) information (ALT), and quality (QUAL) are information that are important for detecting a cancer diagnostic marker. Among these information, information with respect to a portion having a nucleotide sequence (nucleotide) different from a reference genome in a cancer sample or a normal sample, i.e., a portion having variation in the cancer sample or the normal sample is important information particularly for detecting a cancer-diagnostic marker. In addition, variant positions, genomic variants, or the like, for each sample may be obtained and utilized as needed.

With respect to information with respect to the portion with variation, chromosome information (#CHROM), in-chromosome nucleotide sequence (nucleotide) position information (POS), reference genome sequence (nucleotide) information (REF), and sample genome sequence (nucleotide) information (ALT) are specifically described as follows. The chromosome information (#CHROM) with respect to the portion with variation is a chromosome in which variation of a nucleotide sequence (nucleotide) occurs when comparing and/or contrasting the whole genome sequence of a cancer sample or a normal sample with reference genome information, the in-chromosome variant positions (POS) is a position of the nucleotide sequence (nucleotide) in which variation occurs in the chromosome corresponding to the chromosome information (#CHROM), the reference genome sequence (nucleotide) information (REF) is a base sequence (nucleotide) of the reference genome corresponding to the same position as the in-chromosome variant positions (POS), and the sample genome sequence (nucleotide) information (ALT) is a nucleotide sequence (nucleotide) present at a position corresponding to the variant positions (POS) in the chromosome.

Referring to FIG. 2, the chromosome information (#CHROM), the in-chromosome variant positions (POS), the reference genome sequence (REF), the sample genome sequence (ALT), and quality (QUAL) are shown in the first line in data of FIG. 2. Values of the chromosome information (#CHROM), the in-chromosome variant positions (POS), the reference genome sequence (REF), the sample genome sequence (ALT), and the quality (QUAL) are shown in the second line in data of FIG. 2. Specifically, the nucleotide sequence of the reference genome at the 109th position (POS) of the chromosome #1 (#CHROM) is ‘A’ (REF), while the nucleotide sequence of the cancer sample and/or the normal sample is T (ALT), which may be determined that the variation occurs, wherein the quality with respect to the variation is 58% (QUAL).

The step of deriving the disease classification ratio (CR) from the analyzed information and sample information is described in detail.

In the step of deriving the disease classification ratio from the analyzed information and sample information, the disease classification ratio for constructing the cancer-specific nucleotide sequence library may be derived.

*The analyzed information corresponds to at least any one or more among the chromosome information (#CHROM), the in-chromosome variant positions (POS), the reference genome sequence (REF), the sample genome sequence (ALT), and the quality information (QUAL) that are obtained by comparing and/or contrasting the whole genome sequence of the cancer sample or the normal sample with reference genome information.

The sample information corresponds to at least one or more among the total number of cancer samples and normal samples, the total number of cancer samples, the total number of normal samples, the number of cancer samples with variation, the number of cancer samples without variation, the number of normal samples with variation, and the number of normal samples without variation.

The disease classification ratio may be derived from any function using the sample information for each nucleotide sequence variation (or a nucleotide variation) as a parameter after identifying nucleotide sequence variation (or variation) of the cancer sample and/or the normal sample based on the analyzed information.

When deriving the disease classification ratio, it is preferable that the number of cancer samples and normal samples is sufficiently secured, and it is preferable to assume a situation where the number of the two samples is not different greatly.

As an example of deriving the disease classification ratio, the disease classification ratio may be derived according to [Equation I] or [Equation II].

$$\begin{matrix}\frac{Number of cancer samples with variations}{Total number of cancer samples} \times \frac{Number of normal samples with variations}{Total number of normal samples} & [ Equation I ] \\ \frac{\begin{matrix}Number of cancer samples with variations + \\ Number of normal samples with variations\end{matrix}}{Total number of samples} & [ Equation II ]\end{matrix}$$

However, since the disease classification ratio is used to construct a library with respect to cancer-specific sequence information in cancer samples and normal samples, functions for obtaining the disease classification ratio may vary depending on the details, shape, size, and the like, of the library to be constructed.

In other words, the function for deriving the disease classification ratio may be arbitrarily determined by a person who practices the present invention according to the analyzed information and the sample information, and is not limited to [Equation I] or [Equation II] below.

In addition, a new disease classification ratio may be derived and used by using the derived disease classification ratio, the analysis information, and sample information.

Calculation of the disease classification ratio value according to [Equation I], in which the number of cancer samples with variation, the number of normal samples without variation, and the total number of samples in the analyzed information and sample information of FIG. 2 described above are used as a parameter, is described as follows. When the variation is positioned at the 109th position of the chromosome #1, a base of the reference genome information is ‘A’, and the ratio of the cancer sample corresponding to ‘T’ is 35/50 (the number of the variations among 50 of the total cancer samples is 35) and the ratio of the normal sample is 20/50 (the number of the variations among 50 of the total cancer samples is 20) in the base of the sample information, the disease classification ratio at the 109th base of chromosome #1 has a value of 0.28 according to [Equation I].

In the method for detecting a cancer-specific diagnostic marker in a genome, if variation occurs even in the genome sequence of the normal sample which is the same as the genomic variants generated in the genome sequence sequencing information of the cancer sample when compared to the reference genome information, there is a high possibility that it does not correspond to a cancer-specific change. Therefore, the number of cancer samples with variation and the number of normal samples without variation in the disease classification ratio may act as a particularly important parameter.

Further, it is preferable to identify positions of the genomes in which nucleotide sequence variation commonly appears in the cancer sample and the nucleotide sequence variation does not appear in the normal sample, and extract the position information and the variation information of the nucleotide sequence regarding this.

The step of constructing the library with respect to the cancer-specific nucleotide sequence from whole genome sequence of the cancer sample and the normal sample using the disease classification ratio is described in detail.

In the step of constructing the library of the cancer-specific nucleotide sequence from the whole genome sequence information of the cancer sample and the normal sample using the disease classification ratio, the library including the cancer-specific genomic variants which is the target of the marker for cancer diagnostic may be constructed. Further, it is possible to derive whether probability of cancer determination is the highest when a certain number of nucleotide sequence variations occur for each library using the information included in the library.

The library for cancer-specific nucleotide sequence may be constructed based on the disease classification ratio. Preferably, the disease classification ratio may be derived, and a set of analysis information (chromosome information (#CHROM), the in-chromosome variant positions (POS), the reference genome sequence (REF), the sample genome sequence (ALT), and quality (QUAL)) corresponding to a specific disease classification ratio value or more among respective disease classification ratio values may be determined as a library that corresponds to the specific disease classification ratio.

In other words, the library for cancer-specific nucleotide sequence may correspond to a set of analysis information arranged based on the specific disease classification ratio in the entire analysis information.

FIG. 4 is an example of constructing a library. After the disease classification ratio is derived according to the analysis information and sample information, a library (left) corresponding to 0.7 or more among the derived disease classification ratio values and a library (right) corresponding to 0.6 or more among the derived disease classification ratio values may be constructed. As described above, after the disease classification ratio is derived, the disease classification ratio is determined, and the set of analysis information (chromosome information (#CHROM), the in-chromosome variant positions (POS), the reference genome sequence (REF), the sample genome sequence (ALT), and quality (QUAL)) satisfying the specific disease classification ratio value or more is determined as the library corresponding to the specific disease classification ratio, thereby constructing the library with respect to the cancer-specific nucleotide sequence. The library constructed as described above may be regarded as a set of analysis information satisfying the specific disease classification ratio or more, and the analysis information differs according to the specific disease classification ratio value.

When the library is constructed as described above, it is preferable that as the disease classification ratio value is higher, the analysis information included in the library is lower, but it is not limited thereto. For example, when the disease classification ratio is derived according to [Equation I] or [Equation II] described above, as the specific disease classification ratio value is higher, the analysis information included in the library is reduced. In order to derive the disease classification ratio, it is preferable to use the number of cancer samples with variation and the number of normal samples without variation as a parameter of the sample information used in the function for deriving the disease classification ratio.

In addition, since the disease classification ratio is derived for each base position and variation of analysis information, it is preferable to construct the library by setting a range based on the disease classification ratio such as above or below the specific disease classification ratio value.

The step of deriving the classification accuracy using the disease classification ratio and the number of bases with variation as the parameter from the constructed library is described in detail.

In the step of deriving the classification accuracy using the disease classification ratio and the number of bases with variation as the parameter from the constructed library, it is possible to obtain the set of analysis information having the highest probability and the variation information as a cancer diagnostic marker to be utilized as a marker.

The whole genome sequencing analysis information aligned according to the disease classification ratio in the library is changed, and when the predetermined number of variations is arbitrarily set in the aligned analysis information, the classification accuracy of the cancer sample and the normal sample is changed according to the predetermined number of variations. Here, as the classification accuracy is higher, it may be regarded as the cancer-specific nucleotide sequence. Therefore, when the classification accuracy of the samples is calculated by using the disease classification ratio and the predetermined number of variations as the parameter, it is possible to obtain the most suitable variation information as the cancer diagnostic marker among the whole genome sequence.

When the disease classification ratio and the number of variations are used as parameters, normal-disease sample classification accuracy may be obtained by applying rand measure (rand index) as an objective function, and the maximum classification accuracy of the library according to the disease classification ratio and the predetermined number of variations may be derived using a numerical analysis program such as a matrix laboratory, or the like.

Specifically, when the disease classification ratio (I) is determined and the predetermined number of variations (T) is arbitrarily set in the analysis information aligned based on the determined disease classification ratio (I), the classification accuracy satisfying the predetermined number of variations T when the disease classification ratio is I according to [Equation III] below may be obtained.

$$( I , T ) = \frac{TP + TN}{TP + FP + TN + FN} \qquad [ Equation III ]$$

(wherein I is a disease classification ratio, T is a predetermined number of variations that are previously set, TP is the number of cases in which the cancer sample is classified as cancer, TN is the number of cases where the normal sample is classified as normal, FP is the number of cases in which the normal sample is classified as cancer, and FN is the number of cases when the cancer sample is classified as normal).

Further, the disease classification ratio (I) and the predetermined number of variations (T) satisfying the highest classification accuracy in the library may be obtained according to Equation (IV) below.

$$( {I}^{*} , {T}^{*} ) = arg {max}_{I , T} \frac{TP + TN}{TP + FP + TN + FN} \qquad [ Equation IV ]$$

(wherein I is a disease classification ratio, and is represented by I* since it is variable,

T is a predetermined number of variations that are previously set and is represented by T* since it is also variable, and the maximum value of T is the total number of variations included in analyzed information aligned according to I.

TP is the number of cases in which a cancer sample is classified as cancer,

TN is the number of cases where the normal sample is classified as normal,

FP is the number of cases in which the normal sample is classified as cancer, and

FN is the number of cases when the cancer sample is classified as normal).

The disease classification ratio and the predetermined number of variations having the highest classification accuracy may be set, and the base information satisfying this may be utilized as a cancer diagnostic marker. By comparing the base information set as the cancer diagnostic marker with the genome information of various samples, it is possible to diagnose cancer by using only the genome information of the sample.

FIG. 6 is described by way of example as follows. As an analysis result of the whole genome sequence of a sample for specific cancer, when the highest classification accuracy is shown when the disease classification ratio (I) is 0.602 or more and the predetermined number of variations (T) is 4, the base information corresponding to 0.602<I and T=4 in the library may be set as the cancer diagnostic marker. When T is 4 or more in the analysis information where the disease classification ratio (I) is 0.602 or more according to the base information detected by the cancer diagnostic marker, it may be determined as a specific cancer. In order to confirm whether or not the cancer is diagnosed based on these results, the variation is checked at the position in the library for arbitrary samples 1, 2, and 3, and the position where the variation occurs is marked. As confirmation results, samples 1 and 2 correspond to the number of variations of 5 and may be diagnosed as cancer, and sample 3 corresponds to the number of variations of 2 and may be diagnosed as normal (see FIG. 6).

When a size of the library is large, it is preferable to perform a process for reducing complexity since it is difficult to calculate the classification accuracy for all subset and complexity increases.

When the size of the library is N, a case where the number of all subsets is 2\hat{ }N appears. Accordingly, when the size of the library increases, it is difficult to calculate the classification accuracy for all subsets, and the complexity increases, and thus it is necessary to reduce the complexity using a heuristic algorithm in order to solve the problem.

For example, with respect to a case where the size of the subset is N, when the size of the set is reduced step by step by preferentially considering only a case where possibility of the marker is confirmed and the possibility is the highest, the total number of cases for the markers to be searched is reduced to N(N+1)/2.

Further, it is preferable to further perform a process for verifying performance of the finally derived cancer marker.

Specifically, the performance of the marker may be verified by substituting a cancer diagnostic marker to a cancer sample or a normal sample not used for detection of cancer diagnostic marker and calculating the classification accuracy.

In addition, since the accuracy of cancer diagnostic marker may increase as more cancer samples and normal samples are used to detect the cancer diagnostic marker, the genome sequence information of the cancer sample or the normal sample used for the verification is preferably used as feedback information capable of improving the accuracy of the cancer diagnostic marker.

In order to further quickly and accurately perform the method for detecting cancer diagnostic marker as described above, the method may further include extracting a target range for a specific cancer.

The extracting of the target range is preferably performed after analyzing the whole genome sequence and the reference genome information of the cancer sample and the normal sample.

When the cancer diagnostic marker is detected by obtaining the whole genome sequence of the cancer sample and the normal sample from the genetic information database, known cancer genes may be extracted into the target range before analyzing the whole genome sequence of the cancer sample and the normal sample and the reference genome information.

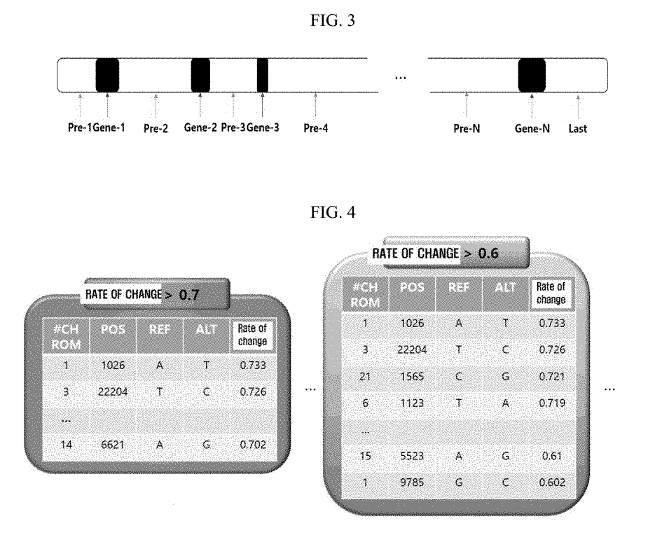

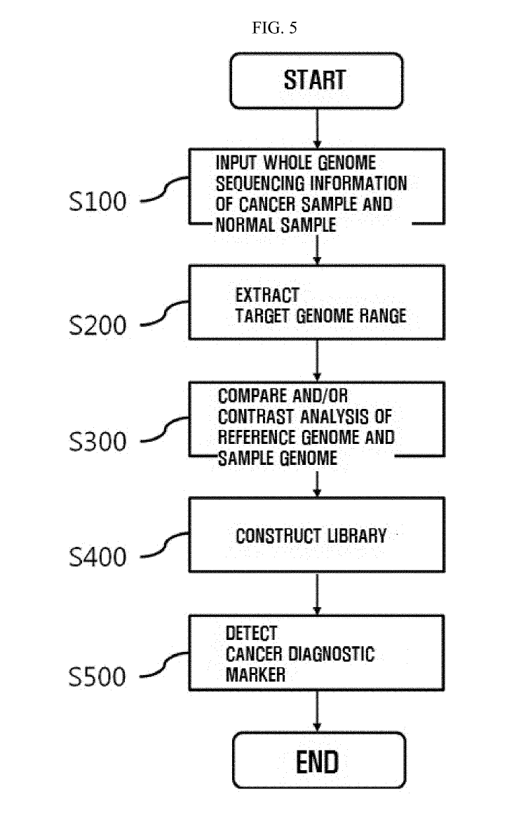

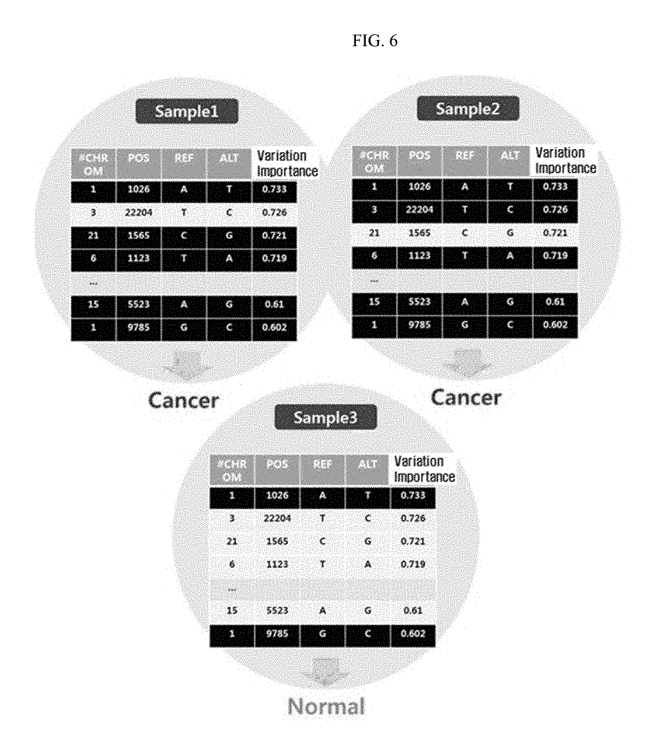

Specifically, the reference genome information, the whole genome sequence of the cancer sample, and the whole genome sequence of the normal sample may be segmented by a preset range as shown in FIG. 3.

The genomic range in which the variation appears may be determined by comparing the whole genome sequence of the segmented cancer samples with the segmented reference genome information.

The genomic range in which the variation appears may be determined by comparing the whole genome sequence of the segmented normal samples with the segmented reference genome information.

When the rate of change of the genome range in which the variation appears is a predetermined rate of change or more, the target genome range for a specific cancer may be extracted by setting the corresponding genome range to the target genome range for a specific cancer. It is preferable to set the predetermined rate of change by comparing the whole genome sequence of the segmented normal samples with the segmented reference genome information, but the present invention is not limited thereto.

In other words, since the whole genome sequence includes not only genome changes due to specific cancers, but also nucleotide sequences varied by an inherent nucleotide sequence and other causes in addition to cancer, it is preferable to extract the genome range that may be regarded as a target of specific cancer.

Here, in the case of the whole genome sequence information, result information which is arranged at the highest probability position by comparing nucleotide sequence fragments having several tens or several hundreds of lengths with the reference genome information. Here, the position of the nucleotide sequence is determined based on the previously stored reference genome information.

Preferably, the reference genome information shown on the upper side of FIG. 1 is previously stored, and has a length of about 3 Gbp.

The numerical information shown at the top represents position information of the reference genome, and in the case of the nucleotide sequence represented by black color below, the numerical information represents the nucleotide sequence of the reference genome.

Further, in the case of the nucleotide sequence fragment represented in the black box shown in the lower part of FIG. 1, as described above, the whole genome sequence of the sample is shown, and the nucleotide sequence fragments having a length of several tens or several hundreds are placed at the highest probability position as compared with the reference genome information. 30 to 40 candidate sequences are averagely present for each one position. Thus, the size of the whole genome sequence data is generally 30 to 40 times larger than the size of the reference genome information, and has a size of about 100 Gbytes. The size thereof may vary depending on the sequencing method.

The size of the sample genome sequence is about 100 Gbytes in size as described above, and thus when all the genomes are compared and analyzed, complexity is very high, which makes it difficult to actualize the implementation.

Thus, the genomes are segmented, and with the segmented genome portions, the cancer sample genome sequencing information or the normal sample genome sequencing information is compared and analyzed with the reference genome information, thereby comparing a change of rage in nucleotide sequence within the segmented genome range.

Here, the rate of change of nucleotide sequence may be defined as a value obtained by segmenting the degree of nucleotide sequence variation by the length of the segmented genome part as compared with the reference genome information in the segmented genome part. In addition, the sequencing information may be used to infer the degree of binding during chemical reaction of the nucleotide sequence fragments using nucleotide sequence variation quality (QUAL) from the sequencing information and the rate of change may be defined based on this change.

In addition, the rate of change may be defined by calculating correlation between the reference genome and cancer sample and normal sample genome in the segmented genome range portion. When the correlation is defined, the nucleotide sequence is cut into words having a predetermined length, and the frequency of the word or the interval where the word having a predetermined length appears is examined, thereby employing PDF correlation. A transition probability of a predetermined length of words may be calculated and then correlation between transition diagrams may be employed.

It is preferable to search a genome segment having a larger rate of change in nucleotide sequence of the cancer sample genome sequencing information as compared to the rate of change in nucleotide sequence of the normal sample genome sequencing information, and define a set of these segments as a target genome range for specific cancer.

The target range extraction is to extract a significant portion of the whole genome with a target genome range for a specific cancer. Based on the position information of the genes, the whole genome may be segmented into gene portions and non-genomic portions.

In detail, it is known so far that the whole genome consists of 23 chromosomes, which is comprised of gene segments and non-genomic segments.

Here, the number of genes is known to be about 25,000 to 30,000, and it is also preferable to include genes that have been newly studied and added.

The target region extraction is to segment the reference genome information, the genome sequencing information of the cancer sample, and the genome sequencing information of the normal sample based on gene position.

As shown in FIG. 3, the segmenting process based on the gene position is performed by assigning a predetermined number according to the order in which each chromosome is positioned, and defining a non-gene portion before the first gene as a pre-1, a non-gene portion between the first gene and the second gene as a pre-2, and a non-gene portion after the last gene as last, thereby segmenting all genome portions.

The method for detecting a cancer diagnostic marker according to the present invention is performed based on the analysis of genome information, it is possible to utilize not only gene but also genomic variation information of non-gene portion, thus detecting cancer diagnostic markers by a method completely different from the conventional method for detecting a cancer diagnostic marker.

Boundaries, lengths, or the like, of genes may be determined according to those that have been studied or known, such as gene analysis information, and the like.

After segmenting, the target genome range of cancer may be extracted by comparing the genome sequencing information of the segmented cancer sample or normal sample with the segmented reference genome information, determining the genomic variants.

Specifically, it is possible to extract portions with variation only by comparing genome sequencing information of the segmented cancer sample or the normal sample with the segmented reference genome information. Here, in the process of extracting portions with variation only, when it is confirmed how much the change occurs in the cancer sample compared with the normal sample for each portion segmented by comparing the rate of change in nucleotide sequence, and a predetermined specific rate of change or more appears in the cancer sample as compared to the normal sample, the corresponding set of genome segments is extracted to be defined as the target genome range for a specific cancer.

In addition, as described above, the rate of change may be defined by calculating the correlation between the reference genome, the cancer sample, and the normal sample genome of the segmented portion.

When the correlation is defined, the nucleotide sequence is cut into words having a predetermined length, and the frequency of the word or the interval where the word having a predetermined length appears is examined, thereby employing PDF correlation. A transition probability of a predetermined length of words may be calculated and then correlation between transition diagrams may be employed.

Further, cancer-specific genomic changes may be extracted by comparing and analyzing the position information and the variation information of the nucleotide sequence defined as a target genome range for a specific cancer.

The above-described method for detecting a cancer diagnostic marker is described again with reference to a flowchart and a specific example shown in FIG. 4. Since the following description is one example for helping understanding of the present invention, a portion of the data processing process and the sample information performed by the program may be omitted and explained using arbitrary value.

As shown in FIG. 4, the method for detecting a cancer-specific diagnostic marker in the genome of the present invention may include an information input step S100, a target range extraction step S200, a comparison analysis step S300, a library construction step S400, and a marker detection step S500. The method for detecting a cancer-specific diagnostic marker in the genome may be in the form of a program executed by an arithmetic processing unit including a computer. In this case, when the cancer sample and the normal sample are directly taken and the whole genome sequence of the cancer sample and the normal sample is input, it is preferable to reverse the order of the target range extraction step (S200) and the comparison analysis step S300.

The information input step S100 may receive whole genome sequence of the cancer sample and whole genome sequence of the normal sample. For example, the National Institutes of Health (NIH) is accredited to receive and input the whole genome sequence for blood cancer, gastric cancer, liver cancer, and normal samples (it is possible to select the number of samples, confirm the sequencing apparatus, and confirm the sequencing method). The whole genome sequence of the cancer sample and the normal sample input at this time may be inputted by downloading the whole genome sequence data of the binary alignment map (BAM) form (see FIG. 2) or by downloading the assembled data based on the reference genome.

Next, the target range extraction step (S200) may extract a target genome range for a specific cancer using the previously stored reference genome information, the whole genome sequence of the cancer sample and the whole genome sequence of the normal sample that are inputted by the information input step S100. For example, in the case of blood cancer, analysis may be performed by extracting about 2,000 genes known to have a high rate of change among these cancer genes as a target range, and together with this, extracting the non-electron portion around the gene with the high rate of change as the target range.

Then, in the analysis step S300, information analyzed by comparing and/or contrasting the whole genome sequence of the cancer sample or the whole genome sequence of the normal sample in the target genome range for the specific cancer extracted by the target range extraction step (S200) (chromosome information (#CHROM), in-chromosome variant positions (POS), reference genome sequence (REF), and sample genome sequence (ALT)) are obtained. Specifically, the chromosome information, the in-chromosome variant positions, nucleotide sequence, quality, and disease classification ratio with respect to the whole genome sequence of the cancer sample with variation or the whole genome sequence of the normal sample may be analyzed.

The chromosome information, the in-chromosome variant positions, nucleotide sequence, quality, and disease classification ratio, in which variation is commonly shown in the whole genome sequence of the cancer sample, may be analyzed.

The chromosome information, the in-chromosome variant positions, nucleotide sequence, quality, and disease classification ratio, in which variation is not commonly shown in the whole genome sequence of the normal sample, may be analyzed to store and manage the variation information of the cancer-specific genome sequence.

In the analysis step, analysis information of the whole genome sequence may be classified and stored using an open source program such as SAMtools, BCFtools, or the like, as a genomic information analysis program as shown in [Table 1] to [Table 5] below. The analysis information is preferably integrated to be used as shown in [Table 5].

TABLE 1
Examples of analyzed information obtained using SAMtools
(QUAL values are not shown separately)

| NUM | #CHROM | POS | REF | ALT | QUAL |
|---|---|---|---|---|---|
| 1 | 1 | 1 | A | A | — |
| 2 | 1 | 2 | C | C | — |
| 3 | 1 | 3 | C | G | — |
| 4 | 1 | 4 | T | T | — |
| 5 | 1 | 5 | A | A | — |
| 6 | 1 | 6 | G | G | — |
| 7 | 1 | 7 | G | G | — |
| 8 | 1 | 8 | A | T | — |
| 9 | 1 | 9 | C | G | — |
| . . . | . . . | . . . | . . . | . . . | . . . |

TABLE 2
Example 1 of analysis information obtained using BCFtools 1

| NUM | #CHROM | POS | REF | ALT | QUAL |
|---|---|---|---|---|---|
| 3 | 1 | 3 | C | G | — |
| 8 | 1 | 8 | A | T | — |
| 9 | 1 | 9 | C | G | — |
| 15 | 1 | 15 | G | C | — |
| . . . | . . . | . . . | . . . | . . . | . . . |

TABLE 3
Example 2 of analysis information obtained using BCFtools

| NUM | #CHROM | POS | REF | ALT | QUAL |
|---|---|---|---|---|---|
| 5232 | 2 | 50 | G | T | — |
| 12033 | 2 | 6851 | C | A | — |
| 12034 | 2 | 6852 | A | T | — |
| 80000 | 3 | 2 | G | A | — |
| 81020 | 3 | 1022 | A | G | — |
| . . . | . . . | . . . | . . . | . . . | . . . |

TABLE 4
Example 3 of analysis information obtained using BCFtools

| NUM | #CHROM | POS | REF | ALT | QUAL |
|---|---|---|---|---|---|
| 8 | 1 | 8 | A | T | — |
| 9 | 1 | 9 | C | G | — |
| 560 | 1 | 560 | T | A | — |
| 562 | 1 | 562 | T | C | — |
| 80000 | 3 | 2 | G | A | — |
| 250080 | 4 | 21 | G | A | — |
| . . . | . . . | . . . | . . . | . . . | . . . |

TABLE 5
Integration of examples of analysis information obtained using BCFtools

| NUM | #CHROM | POS | REF | ALT | QUAL |
|---|---|---|---|---|---|
| 3 | 1 | 3 | C | G | — |
| 8 | 1 | 8 | A | T | — |
| 9 | 1 | 9 | C | G | — |
| 15 | 1 | 15 | G | C | — |
| 560 | 1 | 560 | T | A | — |
| 562 | 1 | 562 | T | C | — |
| 5232 | 2 | 50 | G | T | — |
| 12033 | 2 | 6851 | C | A | — |
| 12034 | 2 | 6852 | A | T | — |
| 80000 | 3 | 2 | G | A | — |
| 81020 | 3 | 1022 | A | G | — |
| 250080 | 4 | 21 | G | A | — |
| . . . | . . . | . . . | . . . | . . . | . . . |

The library construction step (S400) is performed by deriving the disease classification ratio from the analysis information obtained by the analysis step S300 (chromosome information (#CHROM), in-chromosome variant positions (POS), reference genome sequence (REF), sample genome sequence (ALT), and quality (QUAL)), and constructing the library with respect to the whole genome sequence of the cancer sample and the normal sample based on the disease classification ratio.

An arbitrary function is defined as in [Equation I] or [Equation II] above, the disease classification ratio (I) for each base position and variation may be derived from analysis information and sample information, added to analysis information, and summarized as shown in [Table 6].

TABLE 6
Added disease classification ratio (I) to analysis information

| NUM | #CHROM | POS | REF | ALT | I |
|---|---|---|---|---|---|
| 3 | 1 | 3 | C | G | 0.58 |
| 8 | 1 | 8 | A | T | 0.62 |
| 9 | 1 | 9 | C | G | 0.52 |
| 15 | 1 | 15 | G | C | 0.58 |
| 560 | 1 | 560 | T | A | 0.51 |
| 562 | 1 | 562 | T | C | 0.62 |
| 5232 | 2 | 50 | G | T | 0.61 |
| 12033 | 2 | 6851 | C | A | 0.55 |
| 12034 | 2 | 6852 | A | T | 0.54 |
| 80000 | 3 | 2 | G | A | 0.55 |
| 81020 | 3 | 1022 | A | G | 0.65 |
| 250080 | 4 | 21 | G | A | 0.57 |
| . . . | . . . | . . . | . . . | . . . | . . . |

After deriving the disease classification ratio, the library may be constructed based on the disease classification ratio value.

Referring to Table 6, when constructing a library for each disease classification ratio value, it is difficult to determine whether or not the disease is caused because the library is constructed with analysis information corresponding to a single genomic variation.

On the other hand, the analysis information arranged based on the certain disease classification ratio value or more includes one or more variations, and thus it is possible to construct a library having a combination of multiple genomic variations, thereby determining whether or not the disease is caused more accurately.

The analysis information corresponding to the specific disease classification value or more may be aligned as shown in [Table 7] to [Table 10], and the library may be constructed as a set.

TABLE 7
Alignment of analysis information with
disease classification ratio of 0.52 or more

| NUM | #CHROM | POS | REF | ALT | I |
|---|---|---|---|---|---|
| 3 | 1 | 3 | C | G | 0.58 |
| 8 | 1 | 8 | A | T | 0.62 |
| 9 | 1 | 9 | C | G | 0.52 |
| 15 | 1 | 15 | G | C | 0.58 |
| 562 | 1 | 562 | T | C | 0.62 |
| 5232 | 2 | 50 | G | T | 0.61 |
| 12033 | 2 | 6851 | C | A | 0.55 |
| 12034 | 2 | 6852 | A | T | 0.54 |
| 80000 | 3 | 2 | G | A | 0.55 |
| 81020 | 3 | 1022 | A | G | 0.65 |
| 250080 | 4 | 21 | G | A | 0.57 |
| . . . | . . . | . . . | . . . | . . . | . . . |

TABLE 8
Alignment of analysis information with
disease classification ratio of 0.56 or more

| NUM | #CHROM | POS | REF | ALT | I |
|---|---|---|---|---|---|
| 3 | 1 | 3 | C | G | 0.58 |
| 8 | 1 | 8 | A | T | 0.62 |
| 15 | 1 | 15 | G | C | 0.58 |
| 562 | 1 | 562 | T | C | 0.62 |
| 5232 | 2 | 50 | G | T | 0.61 |
| 81020 | 3 | 1022 | A | G | 0.65 |
| 250080 | 4 | 21 | G | A | 0.57 |
| . . . | . . . | . . . | . . . | . . . | . . . |

TABLE 9
Alignment of analysis information with
disease classification ratio of 0.61 or more

| NUM | #CHROM | POS | REF | ALT | I |
|---|---|---|---|---|---|
| 8 | 1 | 8 | A | T | 0.62 |
| 562 | 1 | 562 | T | C | 0.62 |
| 5232 | 2 | 50 | G | T | 0.61 |
| 81020 | 3 | 1022 | A | G | 0.65 |
| . . . | . . . | . . . | . . . | . . . | . . . |

TABLE 10
Alignment of analysis information with
disease classification ratio of 0.62 or more

| NUM | #CHROM | POS | REF | ALT | I |
|---|---|---|---|---|---|
| 8 | 1 | 8 | A | T | 0.62 |
| 562 | 1 | 562 | T | C | 0.62 |
| 81020 | 3 | 1022 | A | G | 0.65 |
| . . . | . . . | . . . | . . . | . . . | . . . |

After the library construction step (S400), the analysis information aligned according to the disease classification ratio (I) is changed, and the predetermined number of variations (T) is specified in the aligned analysis information to obtain the classification accuracy for each predetermined number of variations according to [Equation III].

$$( I , T ) = \frac{TP + TN}{TP + FP + TN + FN} \qquad [ Equation III ]$$

(wherein I is a disease classification ratio, T is a predetermined number of variations that are previously set, TP is the number of cases in which the cancer sample is classified as cancer, TN is the number of cases where the normal sample is classified as normal, FP is the number of cases in which the normal sample is classified as cancer, and FN is the number of cases when the cancer sample is classified as normal).

For example, when the library is constructed based on the disease classification ratio value derived from variation (0.56<1), the analysis information in which the disease classification ratio (I) is 0.56 in the library is the same as the analysis information aligned in [Table 8]. In [Table 8] showing the analysis information when the disease classification ratio (I) is 0.56, the various predetermined numbers of modifications (I) such as T=10, T=20, T=30, and the like may be specified, and the disease-sample classification accuracy may be obtained for each specific predetermined number of mutations (T).

When I is 0.56, and T is 10, classification accuracy: TP+TN/TP+FP+TN+FN=0.75,

When I is 0.56, and T is 20, classification accuracy: TP+TN/TP+FP+TN+FN=0.92

When I is 0.56, and T is 30, classification accuracy: TP+TN TP+FP+TN+FN=0.87

Since the classification accuracy is the highest as 0.92 when I is 0.56 and T is 20, a case where T is 20 for the disease classification ratio (I) of 0.56 corresponds to the variation information that is usable as the most optimal cancer diagnostic marker.

According to this method, base information that may be employed as a cancer diagnostic marker is capable of being detected from the whole genome sequence by obtaining the highest classification accuracy in the whole library according to [Equation IV].

$$( {I}^{*} , {T}^{*} ) = arg {max}_{I , T} \frac{TP + TN}{TP + FP + TN + FN} \qquad [ Equation IV ]$$

(wherein I is a disease classification ratio of nucleotide sequence, and is represented by I* since it is variable,

T is a predetermined number of variations that are previously set, is represented by T* since it is also variable, and the maximum value of T is the total number of variations included in analyzed information aligned according to I,

TP is the number of cases in which the cancer sample is classified as cancer,

TN is the number of cases where the normal sample is classified as normal,

FP is the number of cases in which the normal sample is classified as cancer, and

FN is the number of cases when the cancer sample is classified as normal.)

According to the method for detecting a cancer-specific diagnostic marker in a genome according to an embodiment of the present invention, it is possible to detect a cancer diagnostic marker obtained from whole genome sequence with respect to the cancer sample and the normal sample, and to be applicable to a cancer diagnostic chip, a cancer diagnostic kit, a cancer diagnostic terminal, and a cancer diagnostic system, or the like, to which the detected cancer diagnostic marker is applied. For example, the cancer diagnostic marker is capable of being detected after genomic information of a sample to be detected is acquired by a simple method such as blood sampling, or the like, and thus when the method is applicable to a small-sized medical business such as a biochip, a kit, a terminal device, a system, or the like, a big ripple effect may be achieved on medical industrial fields relating with molecular diagnostics.

In addition, the method for detecting the cancer-specific diagnostic marker of the present invention may compare and analyze genomic variants and variant positions of cancer genomes and normal genomes using genomic nucleotide sequence data obtained from actual cancer patients and normal patients. The analysis information as obtained above may be used to determine cancer-specific genome complex information, thereby deriving the cancer-specific diagnostic marker.

Furthermore, genome information may be additionally acquired over time to identify individual-specific genomic changes. For example, as disease progresses or as the disease is treated from a diseased patient, genetic information may be acquired over a period of time, and analyzed to achieve mapping the disease changes and genome change information.

In addition, sample information having disease and sample information of a region having no disease from one patient may be collected, and genomic information of the two samples may be analyzed, and thus, it is easy to obtain specific-genetic variation information shown in the sample with the disease.

Although the present invention has been described with reference to exemplary embodiments and drawings that are defined with specific matters such as specific components, or the like, these are merely provided to assist general understanding of the present invention, but the present invention is not limited to the disclosed embodiments, and it is obvious that various modifications and changes may be made by those skilled in the art to which the present invention pertains.

### INDUSTRIAL APPLICABILITY

The present invention relates to a method for detecting a cancer-specific diagnostic marker in a genome, and more particularly, to a method capable of detecting cancer-specific genomic changes by identifying the relationship between cancer and genomic variations.

1. A method of detecting a cancer diagnostic marker performed in a program form executed by an arithmetic processing unit including a computer, the method comprising: inputting whole genome sequence of a cancer sample and a normal sample; comparing and/or contrasting the whole genome sequence and the reference genome to obtain analyzed information; deriving a disease classification ratio from the analyzed information and sample information; constructing a library with respect to cancer-specific nucleotide sequence from the whole genome sequence of the cancer sample and the normal sample using the disease classification ratio; and deriving classification accuracy according to the disease classification ratio and a change of the number of variations from the constructed library.

2. The method of claim 1, wherein the information analyzed by comparing and/or contrasting the whole genome sequence and the reference genome of the cancer sample and the normal sample includes chromosome information (#CHROM), in-chromosome variant positions (POS), reference genome sequence (REF), and sample genome sequence (ALT).

3. The method of claim 1, wherein the sample information includes at least one of the total number of cancer samples and normal samples, the total number of cancer samples, the total number of normal samples, the number of cancer samples with variation, the number of cancer samples without variation, the number of normal samples with variation, and the number of normal samples without variation.

4. The method of claim 1, wherein the disease classification ratio is derived by obtaining genomic variants of the cancer sample and/or the normal samples from chromosome information (#CHROM), in-chromosome variant positions (POS), reference genome sequence (REF), and sample genome sequence (ALT) which are information analyzed by comparing and/or contrasting the whole genome sequence of the cancer sample and the normal sample and the reference genome sequence, and employing at least one of the total number of cancer samples and normal samples, the total number of cancer samples, the total number of normal samples, the number of cancer samples with variation, the number of cancer samples without variation, the number of normal samples with variation, and the number of normal samples without variation, which are sample information, as a parameter, for each nucleotide sequence with variation in the cancer sample and/or the normal sample.

5. The method of claim 1, wherein the constructing of the library is performed by obtaining genomic variants of the cancer sample and/or the normal samples from chromosome information (#CHROM), in-chromosome variant positions (POS), reference genome sequence (REF), and sample genome sequence (ALT) which are information analyzed by comparing and/or contrasting the whole genome sequence of the cancer sample and the normal sample and the reference genome, deriving the disease classification ratio employing at least one of the total number of cancer samples and normal samples, the total number of cancer samples, the total number of normal samples, the number of cancer samples with variation, the number of cancer samples without variation, the number of normal samples with variation, and the number of normal samples without variation, which are sample information, as a parameter, for each nucleotide sequence with variation in the cancer sample and/or the normal sample, and constructing the library based on the disease classification ratio derived for each nucleotide sequence with variation.

6. The method of claim 1, wherein the classification accuracy is calculated by obtaining genomic variants of the cancer sample and/or the normal samples from chromosome information (#CHROM), in-chromosome variant positions (POS), reference genome sequence (REF), and sample genome sequence (ALT) which are information analyzed by comparing and/or contrasting the whole genome sequence of the cancer sample and the normal sample and the reference genome, deriving the disease classification ratio by employing at least one of the total number of cancer samples and normal samples, the total number of cancer samples, the total number of normal samples, the number of cancer samples with variation, the number of cancer samples without variation, the number of normal samples with variation, and the number of normal samples without variation, which are sample information, as a parameter, for each base with variation in the cancer sample and/or the normal sample, constructing the library based on the disease classification ratio derived for each base with variation, setting the number of specific variations for each constructed library, and calculating the classification accuracy of the sample for each number of set specific variations.

7. The method of claim 6, wherein the classification accuracy is derived by the following equation according to the disease classification ratio and a change in the number of set specific variations:
$$( {I}^{*} , {T}^{*} ) = arg {max}_{I , T} \frac{TP + TN}{TP + FP + TN + FN}$$
(wherein I is a disease classification ratio of nucleotide sequence, and is represented by I* since it is variable, T is a predetermined number of variations that are previously set, is represented by T* since it is also variable, and the maximum value of T is the total number of variations included in analyzed information aligned according to I, TP is the number of cases in which the cancer sample is classified as cancer, TN is the number of cases where the normal sample is classified as normal, FP is the number of cases in which the normal sample is classified as cancer, and FN is the number of cases when the cancer sample is classified as normal).

8. The method of claim 1, further comprising, after the inputting of the whole genome sequence of the cancer sample and the normal sample, extracting a target genome range for a specific cancer using the inputted whole genome sequence and the reference genome information.
